(19) United States
(12) Patent Application Publication
(10) Pub. No.: US 2012/0004644 A1
(43) Pub. Date: Jan. 5, 2012

(54) PLEURAL DRAINAGE SYSTEM LOCKING DILATOR

(76) Inventors: Griffin Strole, Chicago, IL (US); James Kantola, Waukegan, IL (US); Elise DeVries, Libertyville, IL (US); Anthony M. Looper, Zurich, IL (US); John A. Krueger, Muskego, WI (US)

(21) Appl. No.: 12/827,895

(22) Filed: Jun. 30, 2010

(51) Int. Cl. A61M 27/00

(52) U.S. Cl. 604/540

(57) ABSTRACT

In one embodiment, a locking connector for a pleural access valve comprises an elongated access tip operable for engaging a lumen in a pleural access valve, the elongated access tip defining an internal lumen and the elongated access tip having a radial ridge. The locking connector also comprises a handle section connected with the elongated access tip and substantially concentric to the elongated access tip, the handle section having a first end and a second end, the first end defining an opening in fluid communication with the internal lumen of the elongated access tip, the handle section further comprising at least one support arm extending between the first end and the second end of the handle section. The locking connector also comprises a latch arm connected with the second end of the handle section, the latch arm being substantially concentric and spaced from the elongated access tip, the latch arm defining a notch for engaging a detent on a pleural access valve, wherein the at least one support arm defines an opening and the radial ridge is viewable through the opening.

### BACKGROUND

The present invention generally relates to medical devices utilized in removal of fluid or gases from the pleural or peritoneal cavity of a person. Prior art catheter devices, such as devices manufactured by Denver Biomedical, Inc., implant in the pleural space for extended periods of time and intermittently drain pleural effusion fluids. This prior art catheter has an elongated flexible tube with openings, or fenestrations, along the proximal portion and a self-sealing access valve configuration on the distal end. The fenestrations receive fluids and gases from the pleural space, or cavity. The self-sealing valve end provides an automatic closure of the flow path from the pleural cavity through the catheter and when so closed prevents drainage of fluid from the pleural cavity. In the prior art, a drainage flow path from the pleural cavity begins by insertion of an elongated hollow tub in flow communication with a drainage line into the self-sealing valve configuration of the prior art pleural catheter, thus opening the access valve. The drainage line and access valve are secured together by a snap-fit connection. With the access valve opened by such an elongated hollow tube, negative pressure can be applied to the flow path to remove fluid or gases from the pleural cavity.

While the prior art catheter is effective, some may desire additional functionality. For example, the connection between the drainage line and the catheter may not be considered strong enough because the snap-fit connection may separate if the catheter or drainage line are inadvertently pulled from each other. A need exists to provide a means for a more secure connection between the drainage line and the catheter.

### BRIEF SUMMARY

In one embodiment, a locking connector for a pleural access valve comprises an elongated access tip operable for engaging a lumen in a pleural access valve, the elongated access tip defining an internal lumen and the elongated access tip having a radial ridge. The locking connector also comprises a handle section connected with the elongated access tip and substantially concentric to the elongated access tip, the handle section having a first end and a second end, the first end defining an opening in fluid communication with the internal lumen of the elongated access tip, the handle section further comprising at least one support arm extending between the first end and the second end of the handle section. The locking connector also comprises a latch arm connected with the second end of the handle section, the latch arm being substantially concentric and spaced from the elongated access tip, the latch arm defining a notch for engaging a detent on a pleural access valve, wherein the at least one support arm defines an opening and the radial ridge is viewable through the opening.

In another embodiment, a locking access dilator comprises an elongated tip having an internal lumen and an exterior radial ridge operable for a snap-fit connection with a pleural access valve, the radial ridge also operable to be viewed by a user during a connection with the pleural access valve. The locking access dilator also comprises a latch arm connected to, and spaced from, the elongated tip, the latch arm being substantially concentric and spaced from the elongated access tip, the latch arm defining a notch for engaging a detent on the pleural access valve.

In yet another embodiment, a method of connecting a locking access dilator with a pleural access valve comprises the steps of inserting an elongated access tip into an end of a pleural access valve wherein the elongated access tip opens a duckbill valve within the access valve thereby allowing access to an interior lumen of the access valve. The method further comprises the steps of engaging a ridge of the elongated access tip with a recess within access valve for a snap-fit connection. The method also comprises the steps of rotating the locking access dilator such that a notch in a latch arm of the locking access dilator engages a detent of the pleural access valve. The method also comprises the steps of connecting drainage tubing to the locking access dilator and removing fluid from a pleural cavity.

Advantages of the present invention will become more apparent to those skilled in the art from the following description of the preferred embodiments of the invention which have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 is a perspective view of a prior art pleural catheter that may be used with one embodiment of the locking access dilator of the present invention;

FIG. 2 is a cross sectional view of a prior art pleural catheter that may be used with one embodiment of the locking access dilator of the present invention;

FIG. 3 is a cross-sectional view of the of the locking access dilator according to one embodiment of the present invention;

FIG. 4 is a perspective view of the locking access dilator according to one embodiment of the present invention;

FIG. 5 is a cross-sectional view of the locking access dilator according to one embodiment of the present invention connected with the prior art pleural catheter of FIG. 1;

FIG. 6 is a locking feature of the locking access dilator according to one embodiment of the present invention;

FIG. 7 is a locking access dilator according to one embodiment of the present invention; and

FIG. 8 is a locking access dilator according to one embodiment of the present invention.

### DETAILED DESCRIPTION OF THE DRAWINGS AND THE PRESENTLY PREFERRED EMBODIMENTS

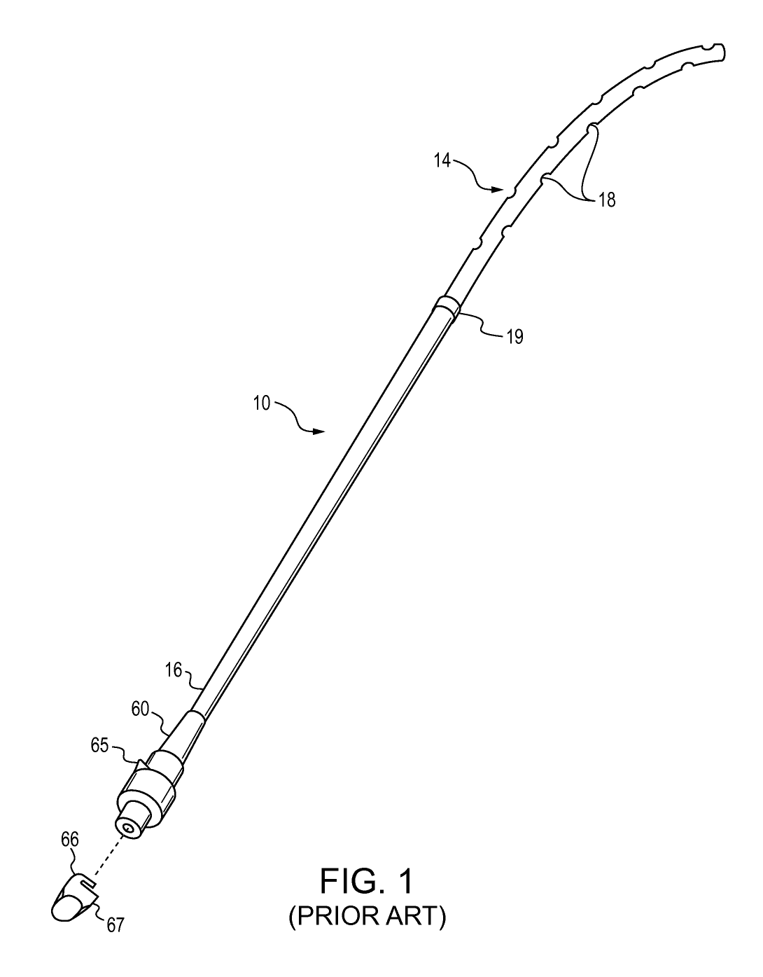

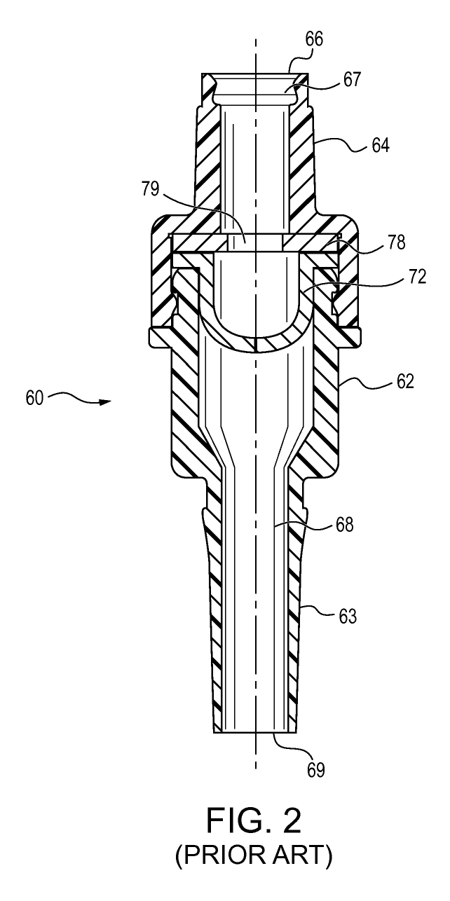

A perspective view of a prior art catheter 10 of the type manufactured by Denver Biomedical, Inc. for use with the present invention is shown in FIG. 1. The depicted prior art catheter 10 has a first end 14 and a second end 16. The first end 14 is operable to be inserted inside a body cavity, such as the pleural cavity. The catheter is implanted into the pleural space using procedures known in the art. The first end 14 is fenestrated with a series of holes 18 allowing fluid communication between the exterior of the catheter and an internal lumen within the catheter 10. Between the first end 14 and the second end 16 is a cuff 19 for placement at the body wall. The second end 16 of the catheter 10 is attached to an access valve 60. A cross-sectional view of the access valve 60 is shown in FIG. 2. As shown in FIG. 1, the access valve 60 also defines a detent 65 projecting radially from the access valve 60. In the prior art, the access valve 60 also engages a cap 67 when the access valve 60 is not in use. The detent 65 of the access valve 60 engages a latch feature 66 of the cap 67 to prevent unintended removal of the cap 67.

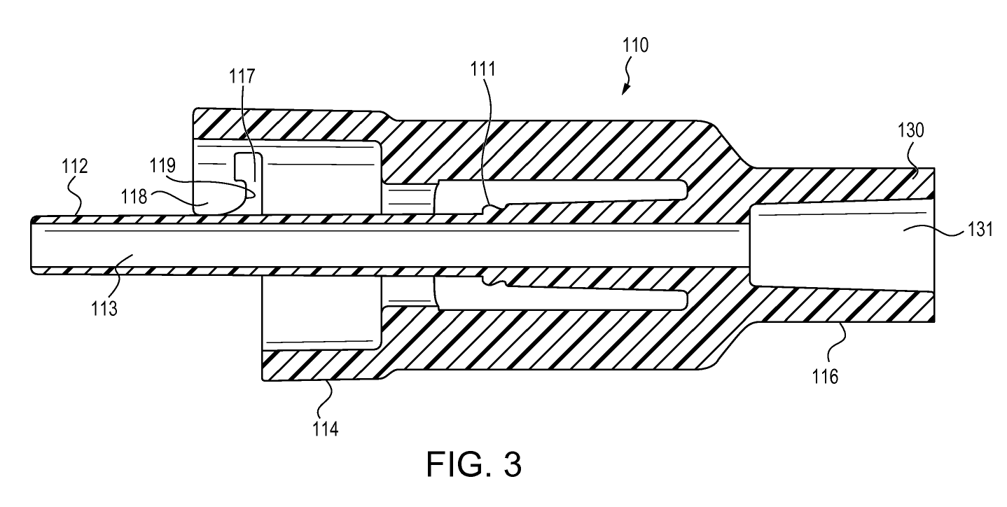

FIG. 2 shows a cross-sectional view of a prior art self-sealing access valve 60 of the type manufactured by Denver Biomedical, Inc. As shown in FIG. 2, the valve includes a body 62 having a distal portion 64 and a proximal portion 63 which are fixedly attached to one another. The end 66 of the distal portion 64 and the end 69 of the proximal portion 63 each define an opening in fluid communication with an internal lumen 68 within the access valve 60. Positioned within this lumen 68 is a “duckbill” valve 72 which is of the type known in the art consisting of an elastomeric, molded, one-piece dome containing a slit in the center of the domed portion. The duckbill valve 72 may be opened by inserting an elongated member through the passageway 68 from the distal portion 64 to pry apart the valve in the manner described below. Adjacent to the duckbill valve 72 toward the distal portion 64 is an elastomeric seal 78. The elastomeric seal 78 is a disk-shaped element having a hole 79 through the center to seal against the outside of the locking access dilator 110 as shown in FIG. 3. At the end 66 of the access valve 60 a radial recess 67 is defined. The radial recess 67 is of a generally circular cross-section and is operable to engage a ridge 111 of the locking access dilator 112 in a snap-fit connection as described below.

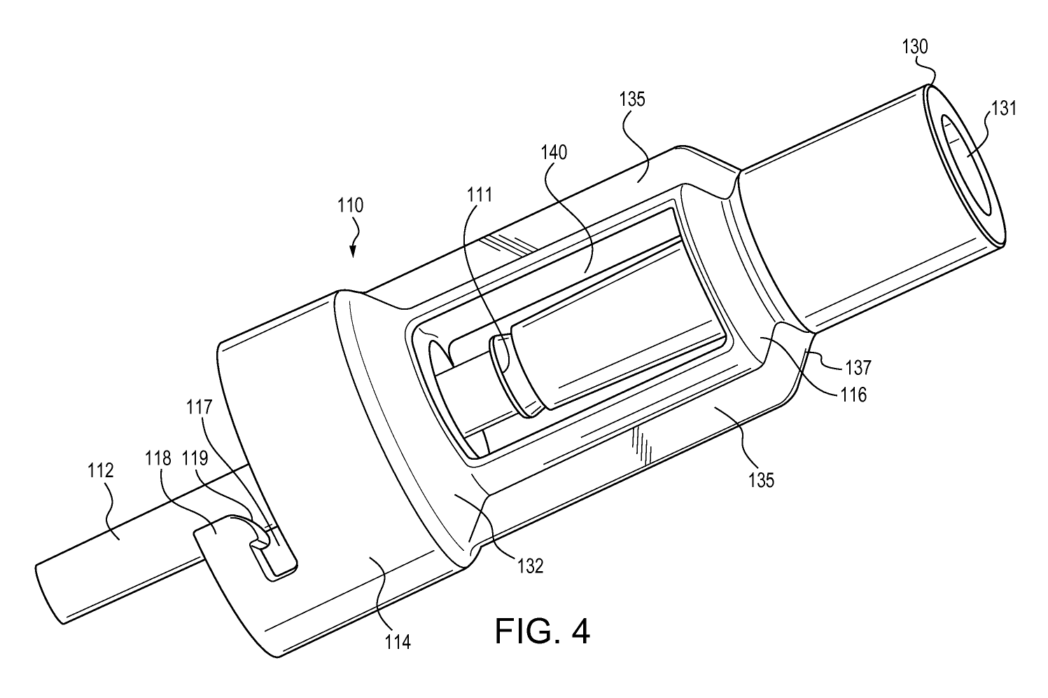

FIG. 3 shows a longitudinal cross sectional view and FIG. 4 shows a perspective view according to one embodiment of the locking access dilator 110. The locking access dilator 110 comprises an elongated access tip 112, a latch feature 114, and a handle portion 116. In one embodiment, the access tip 112, latch feature 114, and handle portion 116 are molded in a single, unitary piece. A single piece design offers a convenient means for manufacturing the locking access dilator 110, as well as advantages over devices have assembled pieces. In another embodiment only the latch feature 114 and handle portion 116 may be molded as a single piece and are overmolded onto the access tip 112. One of skill in the art will understand that other manufacturing methods and combinations may be used.

In one embodiment the locking access dilator 110 is preferably made of the polyeurethane Pellethane 2363-75D manufactured by Lubrizol. In other embodiments, the locking access dilator may also be made of the polyeurethane Isoplast 2510 manufactured by Lubrizol, or any other suitable material for medical applications.

The elongated access tip 112 defines an internal lumen 113 operable for transporting pleural fluids and gases. The diameter of the elongated access tip 112 is sized to be inserted into the opening 66 at the end 64 of the access valve 60 and extend within the internal lumen 68. The elongated access tip 112 is also of sufficient length so that, when connected with the access valve 60, the elongated access tip 112 pierces and separates the duck bill valve 78 of the access valve 60. The elongated access tip 112 also defines a ridge 111 that extends radially outwards upon the exterior of the tip 112. The ridge 111 has a substantially triangular shape in cross section and is operable to provide a snap-fit connection within the radial recess 67 of the access valve 60 of the catheter 10.

Extending generally radially around the elongated access tip 112 is the latch feature 114 of the locking access dilator 110. The latch feature 114 is generally cylindrical and extends radially around, and spaced from, the elongated access tip 112. The latch feature 114 is sized such that it may snugly fit over an end 64 of the access valve 60 when connected. As shown in FIGS. 3-4, the latch feature 114 contains a latch arm 118 that defines a notch 117 in the latch feature 114. The latch arm 118 and notch 117 are sized to accommodate the detent 65 of the access valve 60 when connected. In this embodiment, the latch arm 118 defines a projection 119 for providing a positive stop for locating the detent 65 of the access valve 60 when connected.

The handle portion 116 of the locking access dilator 110 connects the elongated access tip 112 with the latch feature 114 and allows the operator to conveniently manipulate the locking access dilator 110 and ensure a secure connection. The handle portion 116 has a first end 130 and a second end 132. The first end 130 defines an opening 131 that is in flow communication with the lumen 113 of the elongated access tip 112. The elongated access tip 112 is also connected to the handle portion 116 at the first end 130. The handle portion 116 further comprises a plurality of support arms 135 extending to the second end 132 and connecting with the latch feature 114. The support arms 135 are radially spaced from the elongated access tip 112 to allow the access valve 60 to be placed within the locking access dilator 110 when connected.

In a preferred embodiment, two support arms 135 located 180° relative to each other around the longitudinal axis of the elongated access tip 112 are used to support the latch feature 114. In this embodiment, the support arms 135 define two openings 140 through the handle portion 116 so that the operator can view the ridge 111 of the elongated access tip 112 and observe the snap-fit connection with the access valve 60. In this way, the operator can ensure a proper connection with visual confirmation of the snap-fit connection. In one embodiment, the ridge 111 may be colored differently from the elongated access tip 112 and access valve 60. For example, the ridge 111 may be red while the remaining portions of the elongated access tip 112 and the access valve 60 may be white. In this case, the operator can visually confirm a proper snap-fit connection by observing that the red ridge 111 is covered by the white access valve 60. In another embodiment, the plurality of support arms contains fin-like projections 137 on the exterior surface to allow for convenient gripping of the locking access dilator 110 during connection with the access valve 60. The projections 137 allow the operator to better grip the locking access dilator 110 in a manner similar to a wing-nut when rotating the locking access dilator 110 to connect or disconnect the latch arm 118 with the detent 65.

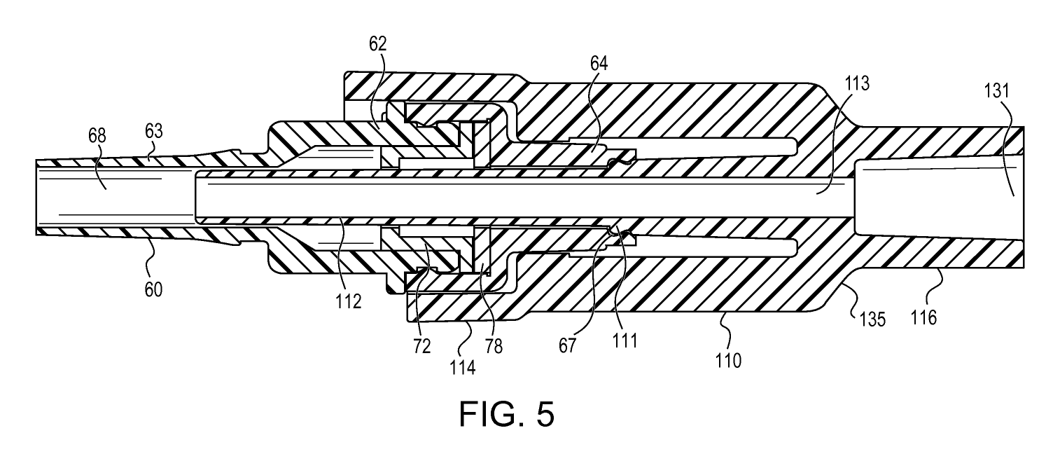

In operation of the locking access dilator 110 with the prior art catheter 10, the elongated access tip 112 of the locking access dilator 110 is inserted into the hollow end 66 of the proximal portion 64 of the body 62 of the self-sealing valve 60 as shown in cross section in FIG. 5. When the locking access dilator 110 is inserted fully into the self-sealing valve 60, the ridge 111 of the elongated access tip 112 engages the matched recess 67 in the proximal portion 64 of the body 62 of the self-sealing valve 60. The locking access dilator 110 can then be rotated such that the notch 117 in the latch arm 118 of the latch feature 114 engages the detent 65 in the proximal portion 64 of the body 62 of the access valve 60, thereby releasably locking the locking access dilator 110 to the access valve 60 of the prior art catheter 10. The elongated access tip 112 of the locking access dilator 110 is slightly larger in its outside diameter than the hole 79 in the elastomeric seal 78 of the self-sealing valve 60, thereby ensuring that a seal is created between the elastomeric seal 78 and the outside of the elongated access tip 112 of the locking access dilator 110 to prevent fluids and gases from leaking. The insertion of the elongated access tip 112 of the locking access dilator 110 into the hollow end 66 of the proximal portion 64 of the body 62 of the self-sealing valve 60 opens the duckbill valve 72 and thereby allows access to the interior of the lumen 68 of the catheter 10. Through a connection to drainage tubing at the handle portion 116 fluid and gas may be removed from the pleural cavity.

The fluid removal procedure is discontinued by rotating the locking access dilator 110 such that the notch 117 in the latch arm 118 of the latch feature 114 disengages the detent 65 on the proximal portion 64 of the self-sealing valve 60, thereby unlocking the locking access dilator 110 from the valve 60 of the prior art catheter 10 and then simply withdrawing the elongated access tip 112 of the locking access dilator 110 from the self-sealing valve 60. As the end of the elongated access tip 112 of the locking access dilator 110 exits the duckbill valve 72, the valve closes and prevents further fluid and gases from flowing out of the self-sealing valve 60.

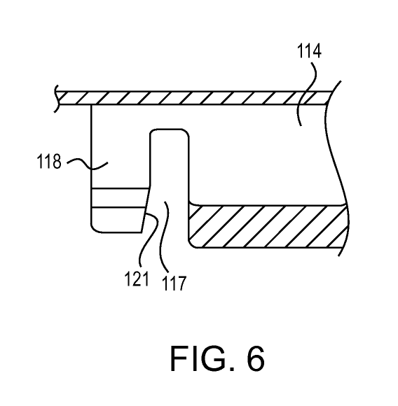

FIG. 6 depicts one embodiment of the latch arm 118 of the latch feature 114. In this embodiment, instead of a projection 119 to provide a positive stop for the detent 65 once connected, the latch arm 118 defines a ramp surface 121 that provides a relatively wider opening to the notch 117. The ramp surface 121 assists the operator in locating and guiding the detent 65 into the notch 117 when the access valve 60 is connected with the locking access dilator 110. In this way, if the detent 65 is misaligned when the detent 65 is rotated into the notch 117, the ramp surface 121 can guide the detent into the notch 117 for proper placement.

According to another embodiment of the latch arm 118, the latch arm 118 can be operable to “break away” from the latch feature if the drainage line is suddenly and unexpectedly pulled away from the catheter. During such an event, the break-away arm will allow the locking access dilator to separate from the access valve 60 without disturbing the catheter from the patient.

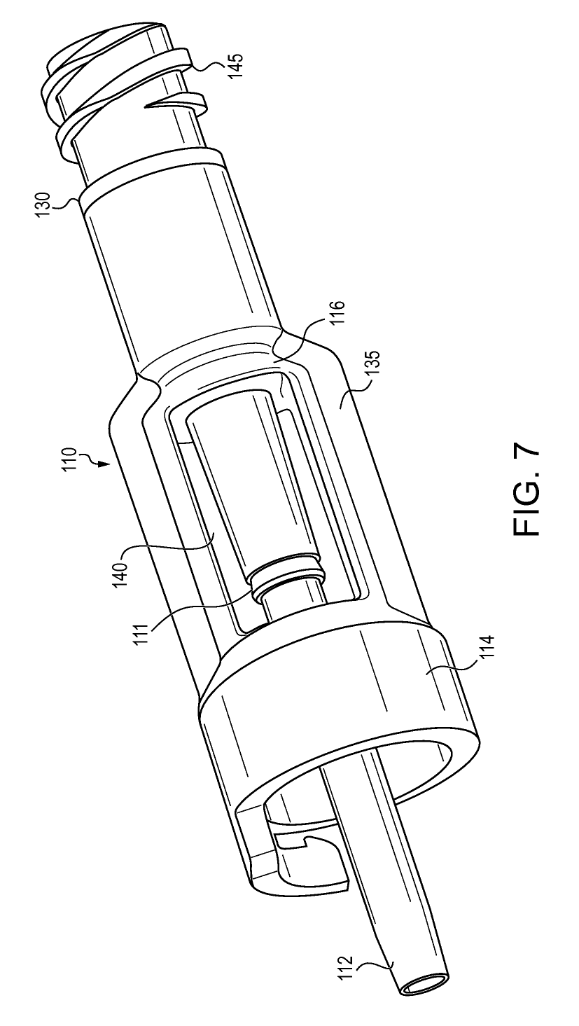

FIG. 7 show another embodiment of the locking access dilator 110 wherein the first end 130 of the handle portion 116 contains a luer-lock type connector 140. In this embodiment, the locking access dilator may be connected to a source of negative pressure through a luer-lock connection. Such a connection allows the locking access dilator 110 to be conveniently removed and replaced in operation and allows the locking access dilator 110 to be conveniently used with a variety of different sources of negative pressure.

Additionally, the luer-lock connector 140 may be used for priming the locking access dilator 110 with sterile fluid, such as with a luer-type connection syringe, prior to connection with the access valve 60. By pre-priming the locking access dilator 110, air can be prevented from entering the catheter 10 and the patient's pleural cavity when the locking access dilator 110 is connected to the access valve 60. Further, the luer-lock connector 140 may be used in conjunction with a luer-type connection syringe for aspirating small amounts of fluid from the catheter line for laboratory testing. In this case, a syringe can be connected with the locking access dilator 110 which can then be used to access the access valve 60. Once the desired amount of fluid is removed by the syringe, the locking access dilator 110 and syringe can be disconnected from the access valve 60.

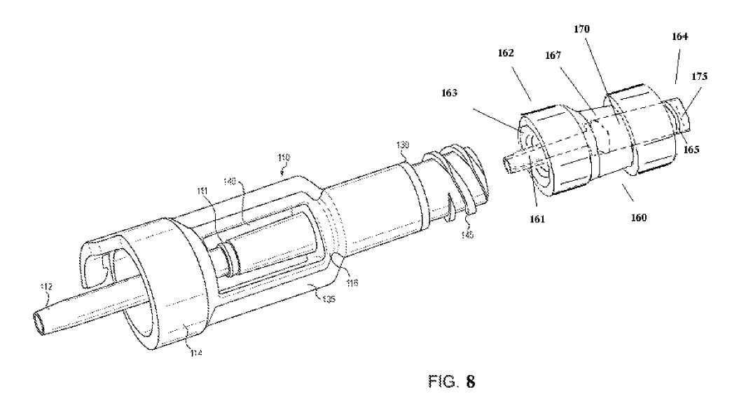

FIG. 8 depicts a luer activated dilator valve 160 that may be used with the locking access dilator 110. In this embodiment, the dilator valve 160 can be threaded onto the locking access dilator 110 to form a sealed end to the catheter line. The dilator valve 160 comprises a first end 162 having a luer-lock type connection 163, a second end 164 having an luer-lock type connection 165, a central chamber 167 and an internal lumen 161 between the first end 162 and second end 164 operable to be in fluid communication with the lumen 113 of the locking access dilator 110 when connected with the locking access dilator 110. The dilator valve 160 also comprises an internal elastomeric septum 170 for providing a selectively sealed end to the lumen 161. The septum 170 contains a slit 175 at the second end 164 of the dilator valve 160. When not in use, the septum 170 extends from the central chamber 167 to within the luer-lock type connection 165 of the second end 164, the central chamber 167 having a larger diameter than the luer-lock type connection 165 of the second end 164. Thus the septum 170 is in radial compression within the luer-lock type connection 165 of the second end 164 relative to the portion of the septum 170 within the central chamber 167.

In operation of this embodiment the first end 162 of the dilator valve 160 can be connected to the luer-lock type connector 140 of the locking access dilator 110. When so connected, the septum 170 provides a sealed end to the locking access dilator 110 and, thus, the catheter line. The lumen 161 of the dilator valve may be accessed by connecting another luer-lock type connector (not shown) to the second end 164 of the dilator valve 160. When connected, the luer-lock type connector will axially compress the end of the septum 170 at the second end 164 and force the slit 175 of the septum 170 into the central chamber 167. When pushed into the relatively larger diameter of the central chamber 167 the septum 170 radially expands and the slit 175 opens, thus providing access to the lumen 161. Removal of the luer-lock type connector at the second end 165 allows the septum 170 to expand axially and compress radially within the luer-lock type connection 165 of the second end 164, thus closing the slit 175 and sealing the catheter line.

It is therefore intended that the foregoing detailed description be regarded as illustrative rather than limiting, and that it be understood that it is the following claims, including all equivalents, that are intended to define the spirit and scope of this invention.

1. A locking connector for a pleural access valve comprising:
an elongated access tip operable for engaging a lumen in a pleural access valve, the elongated access tip defining an internal lumen and the elongated access tip having a radial ridge;
a handle section connected with the elongated access tip and substantially concentric to the elongated access tip, the handle section having a first end and a second end, the first end defining an opening in fluid communication with the internal lumen of the elongated access tip, the handle section further comprising at least one support arm extending between the first end and the second end of the handle section; and
a latch arm connected with the second end of the handle section, the latch arm being substantially concentric and spaced from the elongated access tip, the latch arm defining a notch for engaging a detent on a pleural access valve,
wherein the at least one support arm defines an opening and the radial ridge is viewable through the opening.

2. The locking connector of claim 1 comprising at least two support arms.

3. The locking connector of claim 1 wherein the ridge is colored differently than the elongated access tip.

4. The locking connector of claim 1 wherein the space between the elongated access tip and latch arm is operable to accommodate a pleural access valve.

5. The locking connector of claim 1 wherein the at the least one support arm has a fin for griping the locking connector.

6. The locking connector of claim 1 wherein the latch arm defines a projection operable to provide a positive stop for a detent of an access valve.

7. The locking connector of claim 1 wherein the latch arm defines a ramp surface operable to guide a detent of an access valve into a notch of the latch arm.

8. The locking access connector of claim 1 having only a single latch arm defining a notch for engaging a detent on a pleural access valve.

9. The locking access connector of claim 1 wherein the latch arm is operable to breakaway.

10. The locking access dilator of claim 1 wherein the handle section contains a luer-lock connector.

11. The locking access dilator of claim 1 wherein the elongated access tip, the handle section and the latch arm are unitary.

12. A locking access dilator comprising:
an elongated tip having an internal lumen and an exterior radial ridge operable for a snap-fit connection with a pleural access valve, the radial ridge also operable to be viewed by a user during a connection with the pleural access valve; and
a latch arm connected to, and spaced from, the elongated tip, the latch arm being substantially concentric and spaced from the elongated access tip, the latch arm defining a notch for engaging a detent on the pleural access valve.

13. The locking access dilator of claim 12 wherein the elongated access tip and the latch arm are unitary.

14. The locking access connector of claim 12 having only a single latch arm defining a notch for engaging a detent on a pleural access valve.

15. The locking connector of claim 12 wherein the radial ridge is colored differently that the elongated access tip.

16. The locking connector of claim 12 wherein the space between the elongated access tip and latch arm is operable to accommodate the pleural access valve.

17. A method of connecting a locking access dilator with an pleural access valve comprising the steps of:
inserting an elongated access tip into an end of a pleural access valve wherein the elongated access tip opens a duckbill valve within the access valve thereby allowing access to an interior lumen of the access valve;
engaging a ridge of the elongated access tip with a recess within access valve for a snap-fit connection;
rotating the locking access dilator such that a notch in a latch arm of the locking access dilator engages a detent of the pleural access valve;
connecting drainage tubing to the locking access dilator and removing fluid from a pleural cavity.

18. The method of claim 17 wherein the latch arm defines a projection operable to provide a positive stop for the detent of the access valve.

19. The method of claim 17 wherein the latch arm defines a ramp surface operable to guide the detent of an access valve into the notch of the latch arm.
